(19) United States
(12) Patent Application Publication
(10) Pub. No.: US 2013/0304163 A1
(43) Pub. Date: Nov. 14, 2013

(54) OPTICAL TREATMENT DEVICE FOR SCALP AND HAIR

(71) Applicants: Woisook Moon, Busan (KR); Jinnam Moon, Busan (KR); Myongkyun Roh, Busan (KR)

(72) Inventors: Woisook Moon, Busan (KR); Jinnam Moon, Busan (KR); Myongkyun Roh, Busan (KR)

(73) Assignee: ECOMINE CO., LTD., Busan (KR)

(21) Appl. No.: 13/980,839

(22) PCT Filed: Apr. 26, 2012
(86) PCT No.: PCT/KR2012/003234

§ 371 Date: Jul. 19, 2013

(30) Foreign Application Priority Data
Aug. 1, 2011 (KR) 10-2011-0076505

(51) Int. Cl. A61N 5/06

(52) U.S. Cl. CPC A61N 5/0617
USPC 607/88

(57) ABSTRACT

Provided is an optical treatment device for a scalp and a hair. According to the optical treatment device for the scalp and the hair, LEDs are mounted on a device to irradiate an ultraviolet light and visible light to the scalp and the hair, so that the metabolism of cells is activated. Iontophoresis equipment is mounted at the upper portion of the device or the lateral side portion of the device to apply the potential difference to the skin, thereby changing an electric environment of the skin, so that the transmittance of ionized medicine through the skin is increased. The optical treatment device includes a body, an MTS cartridge detachably provided at one side of the body, and an LED lamp interposed between the body and the MTS cartridge.

### TECHNICAL FIELD

The present invention relates to an optical treatment device for a scalp and a hair. In more detail, the present invention relates to an optical treatment device for a scalp and a hair, which can be used as a common household product since a wide use space and an accessorial special medical device are not required, can be easily carried due to a compact structure, can induce collagen remodeling by stimulating fibroblasts through the physical damage of the scalp by micro-needles based on a micro-needle treatment, can provide a passage of effectively infiltrating medicine, which has the great molecular weight or difficult to be polarized, into the skin, thereby re-generating the skin. According to the optical treatment device for the scalp and the hair of the present invention, LEDs are mounted on a device to irradiate an ultraviolet light and a visible light to the scalp and the hair, so that the metabolism of cells can be activated. Accordingly, the healing and the growing of the damaged cells can be promoted. Iontophoresis equipment is mounted at the upper portion of the device or the lateral side portion of the device to apply the potential difference to the skin, thereby changing an electric environment of the skin, so that the transmittance of ionized medicine through the skin is increased. After inserting a ball-bearing type ample into the device, the ample is allowed to protrude from the upper portion of the device, and rubbed on the skin in such a manner that the medicine is adsorbed onto the skin to discharge the medicine while a message is performed with respect to the scalp with the medicine. Accordingly, the cell organization of the scalp is activated, the growth of the hair is promoted, and the hair loss is prevented, so that the hair and the scalp can be maintained at a healthy state.

### BACKGROUND ART

The present age is referred to as an image age. Modern people meet and communicate together in a social life. Accordingly, it goes without saying that the first impression is very important at a meeting. In this situation, modern people spend a great amount of time and cost to manage their scalps and hairs in order to improve their images and satisfy the demand for various expressions. However, modern people have undergone the scalp and hair damage due to inborn causes such as genetic conditions or posterior causes such as stress or disease.

Trials to use light in order to care a damaged scalp and a damaged hair have been carried out. A laser has been mainly used as a light source in order to use light for treatment. Recently, lasers, which are being used in the department of the dermatology, include CO2 Laser, flashlamp-pumped pulsed dye lasers, argon lasers, copper vapor layers, krypton lasers, and diode lasers.

The lasers have been used to remove a mole, corns, warts, epidermal nevi, freckles having melanin, lentigo, aged spots, ota birthmark, becke's nevus, and tattoo. The lasers for the treatment are mainly classified into lasers having 511, 514, 532, and 1064-nm lasers, and used for the treatment of vascular skin lesion.

However, since the laser treatment is based on the characteristic of photo-thermolysis, cells surrounding a target cell to be treated may be thermally damaged. In addition, the laser may cause hyperpigmentation, or potential herpes infection after the treatment, so that the hypertrophic scar and bacterial infection may be caused. In addition, the laser is expensive, and causes fever.

Up to now, lasers, which have been suggested for the treatment of the scalp and the hair, employ laser diodes (LD) and lamps. However, in the LD, moisture in cells may absorb thermal energy to cause thermal damage to surrounding cells, hyperpigmentation may be caused, and potential herpes infection may be caused, which represent all side effects caused through typical laser treatment. In addition, since the device has a great volume, the device cannot be carried. Further, most devices are fixed to the head of a user or covered on the head of the user, so that the user has a difficulty in periodically utilizing the device in a daily life while carrying the device. In addition, the treatment device for the scalp and the hair prevents only an outer hair from being damaged or treats only the damaged outer hair, and does not directly irradiate laser to an internal hair around a hair root. Accordingly, there is no effect of preventing the scalp and the internal hair from being damaged or treating the damaged internal hair.

### DISCLOSURE

### Technical Problem

Accordingly, the present invention has been made keeping in mind the above problems occurring in the related art, and an object of the present invention is to provide an optical treatment device for a scalp and a hair, which can be used as a common household product since a wide use space is not required, can be easily carried, can provide a passage of effectively infiltrating medicine, which has the great molecular weight or difficult to be polarized, into a skin, to re-generate the skin, can promote the healing and the growing of the damaged cells by activating the metabolism of cells, can increase the transmittance of ionized medicine through the skin, and can activate the cell organization of the scalp, promote the growth of the hair, and prevent the hair loss, so that the hair and the scalp can be maintained at a healthy state.

### Technical Solution

In order to accomplish the above object, there is provided an optical treatment device for a scalp and a hair. The optical treatment device includes a body, an MTS cartridge detachably provided at one side of the body, and an LED lamp interposed between the body and the MTS cartridge.

In addition, the body may include an upper body and a lower body, and a pair of electrodes may be interposed between the upper body and the lower body to make potential difference such that iontophoresis is activated.

Further, the optical treatment device further includes an ion head on which the LED lamp is mounted.

In addition, the body may be provided therein with an ample holder in which an ample is installed.

In addition, the ion head may be provided at a center thereof with an ample ball bearing.

### Advantageous Effect

As described above, according to the present invention, the optical treatment device for the scalp and the hair can be used as a common household product since the wide use space and the accessorial special medical device are not required. The optical treatment device can be easily carried due to a compact structure.

In addition, according to the present invention, collagen remodeling can be induced by stimulating fibroblasts through the physical damage of the scalp by micro-needles based on a micro-needle treatment. A passage of effectively infiltrating medicine, which has the great molecular weight or difficult to be polarized, can be provided into the skin, thereby re-generating the skin.

Further, according to the present invention, LEDs are mounted on a device to irradiate an ultraviolet light and a visible light to the scalp and the hair, so that the metabolism of cells can be activated. Accordingly, the healing and the growing of the damaged cells can be promoted.

In addition, according to the present invention, iontophoresis equipment is mounted at the upper portion of the device or the lateral side portion of the device to apply the potential difference to the skin, thereby changing an electric environment of the skin, so that the transmittance of ionized medicine through the skin is increased. After inserting a ball-bearing type ample into the device, the ample is allowed to protrude from the upper portion of the device, and rubbed on the skin in such a manner that the medicine is adsorbed onto the skin to discharge the medicine while a message is performed with respect to the scalp with the medicine. Accordingly, the cell organization of the scalp is activated, the growth of the hair is promoted, and the hair loss is prevented, so that the hair and the scalp can be maintained at a healthy state.

### DESCRIPTION OF DRAWINGS

FIGS. 1a to 1c are perspective views showing an optical treatment device for a scalp and a hair according to a first embodiment of the present invention.

FIGS. 2a and 2b are perspective views showing an optical treatment device for a scalp and a hair according to a second embodiment of the present invention.

FIGS. 3a to 3c are perspective views showing an optical treatment device for a scalp and a hair according to a third embodiment of the present invention.

FIGS. 4a to 4c are perspective views showing an optical treatment device for a scalp and a hair according to a fourth embodiment of the present invention.

FIGS. 5a to 5c are perspective views showing an optical treatment device for a scalp and a hair according to a fifth embodiment of the present invention.

FIG. 6 is a graph showing optical output power schemes of an LED and an LD and the relationship between the quantity of current and optical power in the LED and the LD.

FIG. 7 is a schematic view showing the procedure of preparing a high-functional ample.

FIG. 8 is a schematic view to explain the procedure of promoting the percutaneous absorption of the high-functional ample.

### BEST MODE

An optical treatment device for a scalp and a hair according to the present invention includes a body, an MTS cartridge detachably provided at one side of the body; and an LED lamp provided between the body and the MTS cartridge.

### MODE FOR INVENTION

Hereinafter, an exemplary embodiment of the present invention will be described in detail with reference to accompanying drawings. The same reference numerals will be used to refer to the same elements throughout the drawings. In a description of the embodiment, if the function or the structure related to the disclosure and generally known to those skilled in the art make the subject matter of the disclosure unclear, the details of the function or the structure will be omitted. It should be understood that, although the exemplary embodiment of the present invention is described hereinafter, the technical spirit of the present invention is not limited thereto, and can have various modifications.

FIGS. 1a to 1c are perspective views showing an optical treatment device for a scalp and a hair according to a first embodiment of the present invention. FIG. 1a is a perspective view showing an optical treatment device for a scalp and a hair having an MTS cartridge attached thereto. FIG. 1b is a perspective view showing an optical treatment device for a scalp and a hair without the MTS cartridge. FIG. 1c is a perspective view showing a body cover.

Referring to FIGS. 1a to 1c, the optical treatment device for the scalp and the hair according to the first embodiment of the present invention includes an upper body 10, a button lock 12, a power button 14, an ample holder 16, an ample cover 18, a battery cover 20, a lower body 22, a left electrode 24, a right electrode 26, an ion head 28, a body top 30, an LED lamp 32, an ample ball bearing 34, an

MTS cartridge 36, and a body cover 38.

The upper body 10 has a streamlined shape with a long length so that a user can easily hold the upper body 10 with the hand of the user. The upper body 10 includes the ample holder 16 having an ample (not shown) to be inserted therein, or to be replaced with new one, the ample cover 18 to close the ample holder 16 after the ample has been inserted or to open the ample holder 16 when the ample is replaced with new on, the button lock 12 to open or close the ample cover 18, and the power button 14 to make the potential difference between electrodes 24 and 26 to activate the LED lamp 32 and iontophoresis.

The button lock 12 is provided at the upper body 10 to actuate the ample cover 18. For example, when the button lock 12 is pressed, the ample cover 18 is open. If the button lock 12 is released after the ample cover 18 has been closed in the state that the button lock 12 is pressed, the ample cover 18 may be closed.

The power button 14 is provided at the upper body to apply voltage to the left and right electrodes 14 and 16, and to apply power to an LED embedded in the body top 30 so that the LED lamp 32 is turned on.

The ample holder 16 is provided at the upper body 10 to ensure a space having a predetermined size so that an ample to provide ionized water for iontophoresis to the scalp can be inserted into the ample holder 16. If the ample is inserted into the ample holder 16, a ball bearing 34 of the ample is exposed through the upper body of the body top 30, so that the internal medicine of the ample is provided to a scalp and a hair of the user when a user bring the ball bearing 34 into contact with the scalp or the hair of the user and rubs.

The ample cover 18 is provided at the upper body 10 to open or close the ample holder 16. The ample cover 18 may be open or closed by the button lock 12.

The battery cover 20 is provided at one end portion of the upper body 10 to open or close a battery insertion port when the battery is inserted or replaced.

The lower body 22 is provided in opposition to the upper body 10 and has a streamlined shape with a long length. The upper body 10 and the lower body 22 have shapes corresponding to each other. The left electrode 24 and the right electrode 26 are mounted between the upper body 10 and the lower body 22.

The left electrode 24 and the right electrode 26 are mounted between the upper body 10 and the lower body 22 to provide potential difference in order to activate iontophoresis. The iontophoresis applies the potential difference to the skin of the user to change the electric environment of the skin, thereby increasing the transmittance of the ionized medicine through the skin. In detail, the iontophoresis is to introduce ionizable essence into melanin cellular layer in a derma through the iontophoresis of DC current, so that the skin can obtain a whitening effect and a regeneration effect. For example, when ionized materials include a vitamin-C essence, the ionized materials prevent pigmentation and activate skin cells. In addition, the ionized materials not only provide the whitening effect through pH balancing of the skin, but also provide functions such as the regeneration of the skin at an acne scar. The iontophoresis deeply infiltrate the ionized materials into the skin. In addition, minerals such as copper (Cu) and zinc (Zn), which are effective ingredients to promote the growth of hair, amino acid, and various vitamins such as Panteon are deeply infiltrated into the skin, so that the hair environment can be improved and the hair loss can be prevented.

The left electrode 24 and the right electrode 26 are supplied with power through the power button 14 to make potential difference therebetween (for example, the left electrode serves as a plus (+) electrode, and the right electrode serves as a minus (-) electrode). The potential difference ionizes the ionized materials to deeply deliver the ionized materials into the skin. Accordingly, the organization of an aged and damaged collagen layer is regenerated, thereby preventing the skin from being aged.

The ion head 28 makes contact with the scalp while grounding the side portion of the device, thereby removing the potential difference across a human body, so that the ionized medicine can be smoothly infiltrated.

The body top 30 is coupled with opposite end portions of the upper body 10 and the lower body 22, so that the upper body 10 is mutually coupled with the lower body 22. In addition, the body top 30 includes the LED lamp and the ample ball bearing 34.

A plurality of LED lamps 32 are provided on the body top 30, so that ultraviolet light and visible light are emitted to the scalp and the hair, thereby providing an LED optical treatment scheme (photodynamic therapy). A red LED light having the wavelength band of about 630 nm activates the metabolism of cells, thereby healing the damaged cells and promoting the growth of the cells. In addition, the red LED light has a wavelength longer than that of a typical medical layer, so that the red LED light can be more deeply infiltrated into the cell organization, and can induce the active generation of the target cell without damaging the peripheral cells.

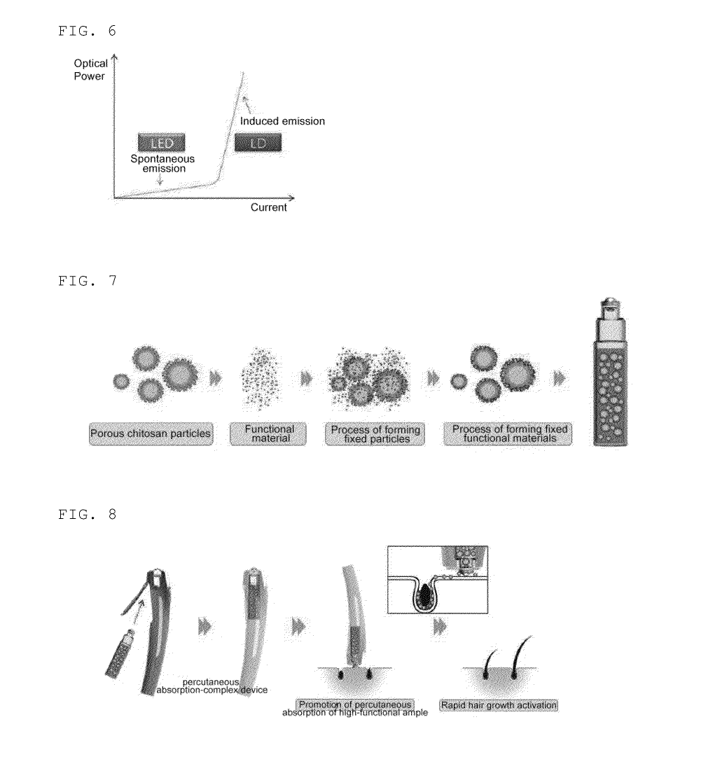

FIG. 6 is a graph showing optical output power schemes of an LED and an LD and the relationship between the quantity of current and optical power in the LED and the LD.

TABLE 1

| | | LD | LED |
|---|---|---|---|
| | | (Laser Diode | (Light-Emitting Diode) |
| Light emission | | Induced emission | Spontaneous emission |
| scheme | | scheme | |
| Wavelength shape | | Wavelength | Dispersion |
| | | having strong | wavelength |
| | | linearity | |
| Energy Level | | High energy | Low energy |
| | | orientation | orientation |
| Absorption type | | Resulting in non- | Non-absorption of |
| of irradiated | | selective absorption | energy in cell |
| light energy on | | of thermal energy | |
| to human body. | | due to moisture | |
| | | in cell (thermal | |
| | | damage is caused to | |
| | | surrounding cells | |
| | | of target cell) | |
| Wavelength region | | Ultraviolet light, | Ultraviolet light, |
| | | Visible light, and | Visible light, and |
| | | Infrared light | Infrared light |
| Use range | 488-514 | used for the | |
| according to | | treatment of | |
| wavelengths | | vascular skin | |
| (nm) | | lesion | |
| | 532 | | |
| | 568 | | |
| | 577~630 | | |
| | 578 | | |
| | 577, 585 | | |
| | 595, 600 | | |
| | 632.8 | Cellular | High cellular |
| | | multiplication | multiplication, |
| | 633 | | injury cure, anti- |
| | | | inflammatory, |
| | 660~950 | | pricking |
| | | | suppression |

| | | |
|---|---|---|
| Side effects | Surrounding cells are | — |
| | thermally damaged. | |
| | hyperpigmentation | — |
| | potential herpes | — |
| | infection | |
| Economic efficiency | Low | High |

The ample ball bearing 34 protrudes from the center of the body top 30. The ample ball bearing 34 discharges the internal medicine of the ample through the surface adsorption scheme in which the ample ball bearing 34 is rubbed on the scalp and the hair.

The MTS (micro-needle therapy system) cartridge 36 is detachably coupled with the body top 30. The MTS is a micro-needle therapy system, and micro-needles cause the physical damage to the skin to stimulate the fibroblast, thereby inducing collagen remodeling and providing a passage in which medicine having the great molecular weight or medicine difficult to be polarized can be effectively infiltrated into the skin to help the skin regenerate.

The MTS provides a process of naturally promoting collagen regeneration at the derma and the stratum basal. The MTS inflicts countless injuries on the skin without damaging the epidermis, so that the injuries are naturally healed and various growth factors are emitted, thereby inducing the generation of new collagen. The passage for the absorption of a new effective ingredient is provided to the skin by a micro-needle passing through a honey layer, so that the whitening ingredients or collagen regeneration ingredients are easily infiltrated into the skin, so that the treatment effect can be maximized. Collagen and cells are continuously and repeatedly regenerated by the effective ingredients stored in the skin through the process.

The body cover 38 is coupled with the MTS cartridge 36 to protect the MTS cartridge 36 when a user carries with the MTS cartridge 36 or stores the MTS cartridge 36.

Hereinafter, the operating method of the optical treatment device for the scalp and the hair according to the first embodiment of the present invention will be described in detail.

First, after attaching the MTS cartridge 36 to the body top 30, micro-needles are applied to the scalp to make a passage for the absorption of the effective ingredients. Naturally, even though the MTS cartridge 36 has been coupled with the body top 30, the body cover 38 is uncovered, so that the MTS procedure may be carried out.

Next, the MTS cartridge 36 is separated from the body top 30, so that the LED LAMP 32 and the ample ball bearing 34 are exposed to the outside. The ample cover 18 is open by using the button lock 12, the ample is inserted into the ample holder 16, and the ample cover 18 is closed. If the power button 14 is pressed, the potential difference is made between the electrodes 24 and 26, thereby ionizing the ionized medicine provided in the ample. If the ample ball bearing 34 is rubbed on the scalp, the ionized medicine is absorbed into the scalp through the iontophoresis.

In addition, the LED lamp 32 is turned on to irradiate light to the scalp, thereby activating the metabolism of the scalp, healing damaged cells, and promoting the growth of the cells. If necessary, a button to apply power to the LED lamp 32 is additionally provided to allow the LED lamp 32 to irradiate light to the scalp, so that the LED optical treatment may be performed before medicine is introduced into the scalp or after the medicine has been introduced into the scalp.

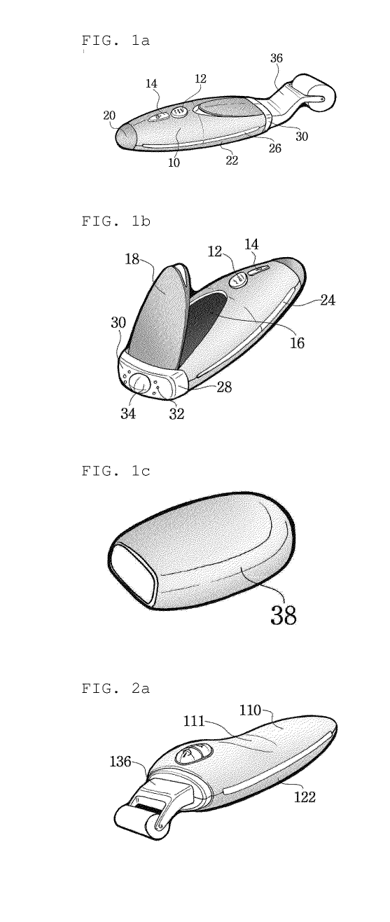

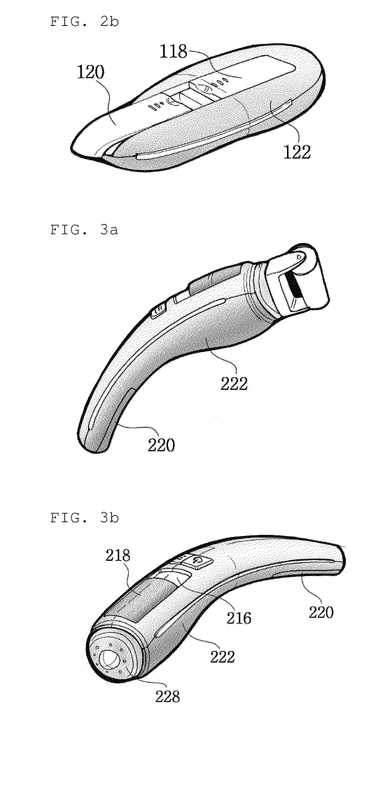

FIGS. 2a and 2b are perspective views showing an optical treatment device for a scalp and a hair according to a second embodiment of the present invention. FIG. 2a is a front perspective view showing the optical treatment device for a scalp and a hair having the MTS cartridge attached thereto. FIG. 2b is a rear perspective view showing the optical treatment device for a scalp and a hair without the MTS cartridge. The second embodiment of the present invention is similar to the first embodiment of the present invention except that the ample holder and the battery insertion port are provided at the lower body, and the whole shape and the shape of the MTS cartridge 136 are modified according to the second embodiment. Accordingly, the second embodiment will be described while focusing on the difference from the first embodiment.

Referring to FIGS. 2a and 2b, the optical treatment device for the scalp and the hair according to the second embodiment of the present invention is provided at a lower body 122 thereof with an ample holder and a battery insertion port. Accordingly, an ample cover 118 and a battery cover 120 are installed in the lower body 122. The ample cover 118 and the battery cover 120 are actuated in a slide scheme.

An upper body 110 of the optical treatment device is provided therein with a hand-holding recess 111 so that the user can easily hold the optical treatment device.

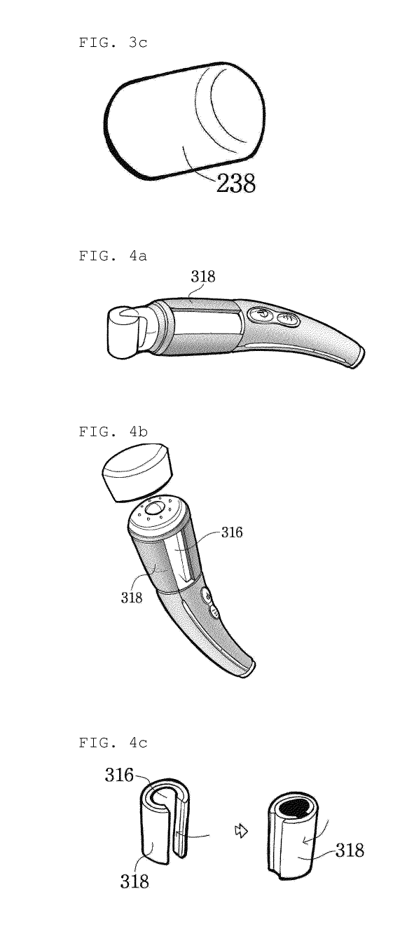

FIGS. 3a to 3c are perspective views showing an optical treatment device for a scalp and a hair according to a third embodiment of the present invention. FIG. 3a is a perspective view showing the optical treatment device for the scalp and the hair having the MTS cartridge attached thereto. FIG. 3b is a perspective view showing the optical treatment device for the scalp and the hair without the MTS cartridge. FIG. 3c is a perspective view showing the body cover. The third embodiment of the present invention is similar to the first embodiment of the present invention except that an ample cover is a transparent window, a battery insertion port is placed at an end portion of a lower body, and the whole shape is modified according to the third embodiment. Accordingly, the third embodiment will be described while focusing on the difference from the first embodiment.

Referring to FIGS. 3a and 3c, the optical treatment device for the scalp and the hair according to the third embodiment of the present invention is made of a transparent window including acryl, so that an amount of medicine remaining in an ample can be checked with the naked eye of the user. In addition, an ample cover 218 is provided in a slide scheme. Accordingly, after inserting the ample into an ample holder 216, the ample cover 218 may slide to close the ample holder 216.

In addition, since the battery insertion port is provided at an end portion of a lower body 222, a battery cover 220 is provided at the end portion of the lower body 222. The ion head 228 is formed in a rectangular shape or a circular shape. Accordingly, a body cover 238 is formed in a circular shape.

FIGS. 4a to 4c are perspective views showing an optical treatment device for a scalp and a hair according to a fourth embodiment of the present invention. FIG. 4a is a perspective view showing the optical treatment device for the scalp and the hair having an MTS cartridge attached thereto. FIG. 4b is a perspective view showing the optical treatment device for the scalp and the hair without the MTS cartridge. FIG. 4c is a perspective view to explain the operating scheme of the ample cover.

The fourth embodiment of the present invention is similar to the first embodiment of the present invention except that an ample holder is open or closed as an ample cover is rotated, and the whole shape is modified according to the fourth embodiment. Accordingly, the fourth embodiment will be described while focusing on the difference from the first embodiment.

Referring to FIGS. 4a and 4c, in the optical treatment device for the scalp and the hair according to the fourth embodiment of the present invention, an ample holder 316 is open or closed by the position relation between the ample holder 316 and an ample cove 318. Referring to FIG. 4c, the ample cover 318 may surround the ample holder 316 to rotate about the ample holder 316. Accordingly, when the ample is inserted, the ample holder 316 is open by rotating the ample cover 318 as shown in the left of FIG. 4C. After the ample has been inserted, the ample holder 316 is closed by rotating the ample cover 318 as shown in the left of FIG. 4C.

In addition, the ample cover 318 includes a transparent material such as acryl so that an amount of medicine remaining in the ample can be checked.

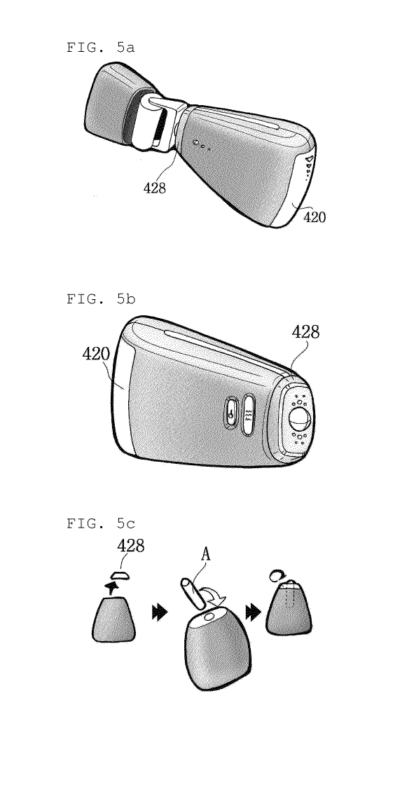

FIGS. 5a to 5c are perspective views showing an optical treatment device for a scalp and a hair according to a fifth embodiment of the present invention. FIG. 5a is a perspective view showing the optical treatment device for the scalp and the hair having an MTS cartridge attached thereto. FIG. 5b is a perspective view showing the optical treatment device for scalp and hair without the MTS cartridge. FIG. 5c is a perspective view to explain an ample inserting scheme. The fifth embodiment of the present invention is similar to the first embodiment of the present invention except that the ample is inserted by opening an ion head, a battery cover is placed at the lower end of the body, and the whole shape is modified. Accordingly, the fifth embodiment will be described while focusing on the difference from the first embodiment.

Referring to FIGS. 5a and 5c, in the optical treatment device for the scalp and the hair according to the fifth embodiment of the present invention, after inserting an ample A by opening an ion head 428, the ion head 428 is closed, thereby completing the ample A. Although the fifth embodiment is realized in the form of an electric razor for a male, a battery cover 420 is placed at a lower end of a body, so that the space arrangement of components can be sufficiently achieved in spite of a small size.

The ion head 428 is open or closed as the ion head 428 rotates.

FIG. 7 is a schematic view showing the procedure of manufacturing a high-functional ample. FIG. 8 is a schematic view to explain the procedure of promoting the percutaneous absorption of the high-functional ample.

Referring to FIG. 7, the high-functional ample inserted into the optical treatment device for the scalp and the hair according to the present invention is prepared by mixing porous chitosan particles with functional materials so that the functional materials are fixed to the porous chitosan particles.

As shown in FIG. 8, hair follicle is activated through the percutaneous absorption of the ample after inserting the high-functional ample according to the preparing method shown in FIG. 7 into the optical treatment device for the scalp and the hair, thereby inducing the rapid growth of hair.

Although a preferred embodiment of the present invention has been described for illustrative purposes, those skilled in the art will appreciate that various modifications, additions and substitutions are possible, without departing from the scope and spirit of the invention as disclosed in the accompanying claims.

### INDUSTRIAL APPLICABILITY

The present invention is extensively applicable to fields of designing and manufacturing a medical optical treatment device to improve scalp and prevent hair loss.

1. An optical treatment device for scalp and hair, the optical treatment device comprising:
a body;
an MTS cartridge detachably provided at one side of the body; and
an LED lamp interposed between the body and the MTS cartridge.

2. The optical treatment device of claim 1, wherein the body comprises an upper body and a lower body, and a pair of electrodes are interposed between the upper body and the lower body to make potential difference such that iontophoresis is activated.

3. The optical treatment device of claim 1, further comprising an ion head on which the LED lamp is mounted.

4. The optical treatment device of claim 1, wherein the body is provided therein with an ample holder in which an ample is installed.

5. The optical treatment device of claim 3, wherein the ion head is provided at a center thereof with an ample ball bearing.
